(19) United States
(12) Patent Application Publication
(10) Pub. No.: US 2017/0304567 A1
(43) Pub. Date: Oct. 26, 2017

(54) INHALATION DEVICE, SYSTEM AND METHOD

(71) Applicant: RESOLVE DIGITAL HEALTH INC., Vaughan (CA)

(72) Inventor: Robert Adelson, Vaughan (CA)

(73) Assignee: RESOLVE DIGITAL HEALTH INC., Vaughan (CA)

(21) Appl. No.: 15/494,339

(22) Filed: Apr. 21, 2017

Related U.S. Application Data
(60) Provisional application No. 62/326,449, filed on Apr. 22, 2016.

(51) Int. Cl. A61M 15/00; A61M 11/04

(52) U.S. Cl. CPC A61M 15/0028; A61M 11/041; A61M 15/0021; A61M 2205/502; A61M 2205/3606; A61M 2205/3584; A61M 2205/3331; A61M 2205/18; A61M 2202/064; A61M 2205/52; A61M 2205/60; A61M 2205/3553

(57) ABSTRACT

A device, system, and method for vaporizing substances in a pod, utilized for inhalation by a user. The device includes components to measure the content and dosage pods containing substances, among other features. The device includes a graphical user interface that may be integrated with the device or implemented through a mobile device, which allows the user to transmit user data to a remote database and/or healthcare provider for personal and clinical data collection. The user data can be used for enhancing personal treatment or clinical research.

### FIELD OF THE INVENTION

The present invention relates generally to inhalation device(s), system(s) and method(s) of operation thereof. More specifically, the present invention relates to device(s), system(s) and method(s) for inhaling vaporized substances contained in a pod.

### BACKGROUND OF THE INVENTION

An inhalation device generally refers to a device that delivers doses of one or more chemicals, herbs, medicines, and/or ingredients (collectively, “substances”) via an inhaled vapor. Inhalation may generally be considered a fast and efficient means of introducing substances into the bloodstream of the user.

Accordingly, inhalation of substances (e.g. medications) may be a desirable delivery route for patients who desire quick relief, or other persons who wish to experience the effects of herbal substances in the body. In the past, substances were typically ignited in order to effect the transformation of compounds into an inhalable state of matter. Once transformed, a substance could be inhaled through the mouth such that it eventually enters the blood-stream via the lungs. To facilitate this process, it may have been common practice to roll a desired substance into a flammable substrate and inhale the desired substance with the aid of a mouthpiece. In this fashion, the practice of smoking became a very popular phenomenon.

In recent years, evidence linking smoking with various cancers has been reported. Smoking has been shown to exacerbate other illnesses and cause a number of undesirable health effects. A desire to limit or prevent these ill health effects has risen in society, among individuals and governments alike. However, complete abstinence may not be the optimal solution considering, for one, the value of inhalation as a route for the administration of substances. The value of this route of delivery has prompted the development of alternate and/or safer technologies to administer substances by inhalation.

Specifically, the realization that inhalable substances can be generated through methods other than ignition have resulted in the development of devices that use various mechanisms to effect vaporization, ionization, or other transformations of desirable substances. However, such conventional devices suffer from the limitation that when used for grinding and heating dried substances and processing the essential extract and vaporizing it, it turns out to be a less effective way of promoting high concentrations of active substances in herbal medicine for inhalation. The manufacturing process to prepare the dried cannabis in the prior art may have been complex and expensive. Further, the lack of sterilization of the substances used in devices of the prior may also be a concern. Specifically, doses of the substances are not provided in a sterilized container or format. Accordingly, there may be potential for the growth of viruses, bacteria and fungus on the substances.

Apart from effectively and safely creating an inhalable state of matter, several further issues remain for physicians' wanting to assist patients using inhaled medication. First, physicians, and thereby patients, currently experience issues with respect to how to determine the effect of a medication on the patient who is self-medicating outside of the physician's direct observation. Further, valuable patient information may not be collected or shared with appropriate parties for further research and development in the area of inhalable medications.

What may be needed is a device and method that provides information on how much of a vaporized substance a user is inhaling, delivers sterile substances, monitors user status (e.g. before, during and/or following an inhalation), disseminates user information (e.g. other users, physicians, support system, database) and/or allows third parties to conduct research on dosing effectiveness, substances and lifestyle information.

There is therefore a need in the art for an inhalation device and method that can overcome the deficiencies associated with the prior art.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written

### OBJECTS OF THE INVENTION

An object of the present disclosure is to provide an inhalation device that can assist self-medicating users in determining the effect of medication outside of physician's direct observation.

An object of the present disclosure is to provide an inhalation device that can collect and disseminate valuable patient information to third parties for further research and development in inhalable medications.

An object of the present disclosure is to provide an inhalation device that can deliver sterile substances.

An object of the present disclosure is to provide an inhalation device that can render information about the amount of vaporized substance inhaled by a user.

An object of the present disclosure is to provide an inhalation device that can monitor a user during different stages of inhalation.

An object of the present disclosure is to provide an efficient and cost-effective inhalation device.

### SUMMARY

The present invention relates generally to inhalation device(s), system(s) and method(s) of operation thereof. More specifically, the present invention relates to device(s), system(s) and method(s) for inhaling vaporized substances contained in a pod.

An aspect of the present disclosure relates to a device for use in association with a container containing a substance for delivery to a user, the device comprising: a vaporizing section adapted to receive the container and vaporize the substance; an inhalation section adapted to deliver the vaporized substance to the user; and an electronics section configured to perform (i) controlling the operation of the device, and (ii) generating user data associated with the delivery of the substance to the user. In an embodiment, the vaporizing section includes a heater for vaporizing the substance. In another embodiment, the vaporizing section is adapted to receive at least two containers. In an embodiment, the device includes a vapor sensor for detecting and/or measuring composition and flow of air and/or vapor moving through the device. In an embodiment, the inhalation section of the device further includes a cooling coil for decreasing vapor temperature. In an embodiment, the heater of the device achieves a predetermined temperature either instantaneously or over a predetermined period of time. In an embodiment, any or a combination of the vaporizing section and the inhalation section further include(s) a perforator to facilitate air circulation within the container. In an embodiment, the device further includes an intake port for drawing-in air external to the device. In an embodiment, the container is a pod.

Another aspect of the present disclosure relates to a system for delivery of a substance contained in a container to a user, the system comprising: at least one device for vaporizing the substance contained in the container, wherein said at least one device comprises at least one processor operative to effect any or a combination of (a) controlling vaporization rate of the substance and (ii) collecting and transmitting user data associated with delivery of the vaporized substance; and a database configured to electronically store the user data associated with delivery of the vaporized substance. In an embodiment, the at least one device further includes geographic tracking means operative to obtain location data of the user. In an embodiment, the at least one device further includes any or a combination of an accelerometer and a gyroscope operative to measure and/or determine the position of the device and to automatically associate the accelerometer data and/or gyroscope data with the device and/or the user. In an embodiment, the at least one device includes a graphical user interface for presenting the user data. In an embodiment, the processor is operative to electronically generate an alert. In an embodiment, the at least one device further includes an external device for receiving user data from the device and/or sending external device data to the device. In an embodiment, the processor of the system is operative to electronically generate a report based on the user data. In an embodiment, the processor of the system is operative to electronically control activation of the heater and temperature of the heater. In an embodiment, the device of the system further includes a reader operative to read a machine readable code associated with the container to collect data associated therewith. In an embodiment, the container is a pod.

Still another aspect of the present disclosure relates to a method for delivering a substance contained in a container to a user, said method comprising the steps of: (a) loading said container into a device; and (b) operating said device to effect vaporization of said substance contained in the container to enable delivery thereof to the user, wherein, said device is operatively coupled to at least one processor, and wherein said at least one processor is configured to (i) control vaporization of the substance and (ii) collect and/or transmit data associated with the delivery of the vaporized substance to the user. In an embodiment, the container is a pod.

Other advantages, features and characteristics of the present disclosure, as well as methods of operation and functions of the related elements of the system, method, device and computer readable medium, and the combination of steps, parts and economics of manufacture, will become more apparent upon consideration of the following detailed description and the appended claims with reference to the accompanying drawings, the latter of which are briefly described hereinbelow.

### BRIEF DESCRIPTION OF THE DRAWINGS

The novel features which are believed to be characteristic of the device according to the present disclosure, as to their structure, organization, use, and method of operation, together with further objectives and advantages thereof, will be better understood from the following drawings in which an embodiment of the disclosure will now be illustrated by way of example. It is expressly understood, however, that the drawings are for the purpose of illustration and description only, and are not intended as a definition of the limits of the invention.

FIG. 1 illustrates an exemplary section view of an inhalation device realized in accordance with an embodiment of the present disclosure.

FIGS. 2A-D illustrate exemplary top, side, rear and front perspective views, respectively, of the inhalation device in accordance with embodiments of the present disclosure.

FIG. 3 illustrates an exemplary front perspective view of the pods for use with the inhalation device in accordance with an embodiment of the present disclosure.

FIGS. 4A-B illustrate side and perspective views, respectively, of the inhalation device in accordance with an embodiment of the present disclosure.

FIGS. 5A-D illustrate screenshots of a digital health infrastructure application in accordance with embodiments of the present disclosure.

### DETAILED DESCRIPTION

The description that follows, and the embodiments described therein, is provided by way of illustration of an example, or examples, of particular embodiments of the principles and aspects of the present disclosure. These examples are provided for the purposes of explanation, and not of limitation, of those principles and of the disclosure.

It should also be appreciated that the present disclosure can be implemented in numerous ways, including as a system, a method or a device. In this specification, these implementations, or any other form that the invention may take, may be referred to as processes. In general, the order of the steps of the disclosed processes may be altered within the scope of the invention.

The drawings are not necessarily to scale and in some instances proportions may have been exaggerated in order to more clearly depict certain embodiments and features of the invention.

In this disclosure, a number of terms and abbreviations are used. The following definitions of such terms and abbreviations are provided.

As used herein, a person skilled in the relevant art may generally understand the term “vaporization” or “vaporize” to generally mean the phase transition of an element, compound or composition etc. from a liquid or a solid (including moisture on the solid) phase to a vapor; with no burning, the absence of fire, smoke and the by-products of burning.

As used herein, a person skilled in the relevant art may generally understand the term “vapor” to generally mean a substance in the gas phase at a temperature lower than its critical point. It may also include one or more substances in a suspension of finely divided solid particles or liquid droplets in a gas, including aerosols, mists and the like.

As used herein, a person skilled in the relevant art may generally understand the term “comprising” to generally mean the presence of the stated features, integers, steps, or components as referred to in the claims, but that it does not preclude the presence or addition of one or more other features, integers, steps, components or groups thereof.

The present invention relates generally to inhalation device(s), system(s) and method(s) of operation thereof. More specifically, the present invention relates to device(s), system(s) and method(s) for inhaling vaporized substances contained in a pod.

An aspect of the present disclosure relates to a device for use in association with a container containing a substance for delivery to a user, the device comprising: a vaporizing section adapted to receive the container and vaporize the substance; an inhalation section adapted to deliver the vaporized substance to the user; and an electronics section configured to perform (i) controlling the operation of the device, and (ii) generating user data associated with the delivery of the substance to the user. In an embodiment, the vaporizing section includes a heater for vaporizing the substance. In another embodiment, the vaporizing section is adapted to receive at least two containers. In an embodiment, the device includes a vapor sensor for detecting and/or measuring composition and flow of air and/or vapor moving through the device. In an embodiment, the inhalation section of the device further includes a cooling coil for decreasing vapor temperature. In an embodiment, the heater of the device achieves a predetermined temperature either instantaneously or over a predetermined period of time. In an embodiment, any or a combination of the vaporizing section and the inhalation section further include(s) a perforator to facilitate air circulation within the container. In an embodiment, the device further includes an intake port for drawing-in air external to the device. In an embodiment, the container is a pod.

Another aspect of the present disclosure relates to a system for delivery of a substance contained in a container to a user, the system comprising: at least one device for vaporizing the substance contained in the container, wherein said at least one device comprises at least one processor operative to effect any or a combination of (a) controlling vaporization rate of the substance and (ii) collecting and transmitting user data associated with delivery of the vaporized substance; and a database configured to electronically store the user data associated with delivery of the vaporized substance. In an embodiment, the at least one device further includes geographic tracking means operative to obtain location data of the user. In an embodiment, the at least one device further includes any or a combination of an accelerometer and a gyroscope operative to measure and/or determine the position of the device and to automatically associate the accelerometer data and/or gyroscope data with the device and/or the user. In an embodiment, the at least one device includes a graphical user interface for presenting the user data. In an embodiment, the processor is operative to electronically generate an alert. In an embodiment, the at least one device further includes an external device for receiving user data from the device and/or sending external device data to the device. In an embodiment, the processor of the system is operative to electronically generate a report based on the user data. In an embodiment, the processor of the system is operative to electronically control activation of the heater and temperature of the heater. In an embodiment, the device of the system further includes a reader operative to read a machine readable code associated with the container to collect data associated therewith. In an embodiment, the container is a pod.

Still another aspect of the present disclosure relates to a method for delivering a substance contained in a container to a user, said method comprising the steps of: (a) loading said container into a device; and (b) operating said device to effect vaporization of said substance contained in the container to enable delivery thereof to the user, wherein, said device is operatively coupled to at least one processor, and wherein said at least one processor is configured to (i) control vaporization of the substance and (ii) collect and/or transmit data associated with the delivery of the vaporized substance to the user. In an embodiment, the container is a pod.

Still another aspect of the present disclosure relates to a method for delivering a substance contained in a pod to a user, said method comprising the steps of: (a) loading said pod into a device; and (b) operating said device to effect vaporization of said substance contained in the pod to enable delivery thereof to the user, wherein, said device is operatively coupled to at least one processor, and wherein said at least one processor is configured to (i) control vaporization of the substance and (ii) collect and/or transmit data associated with the delivery of the vaporized substance to the user.

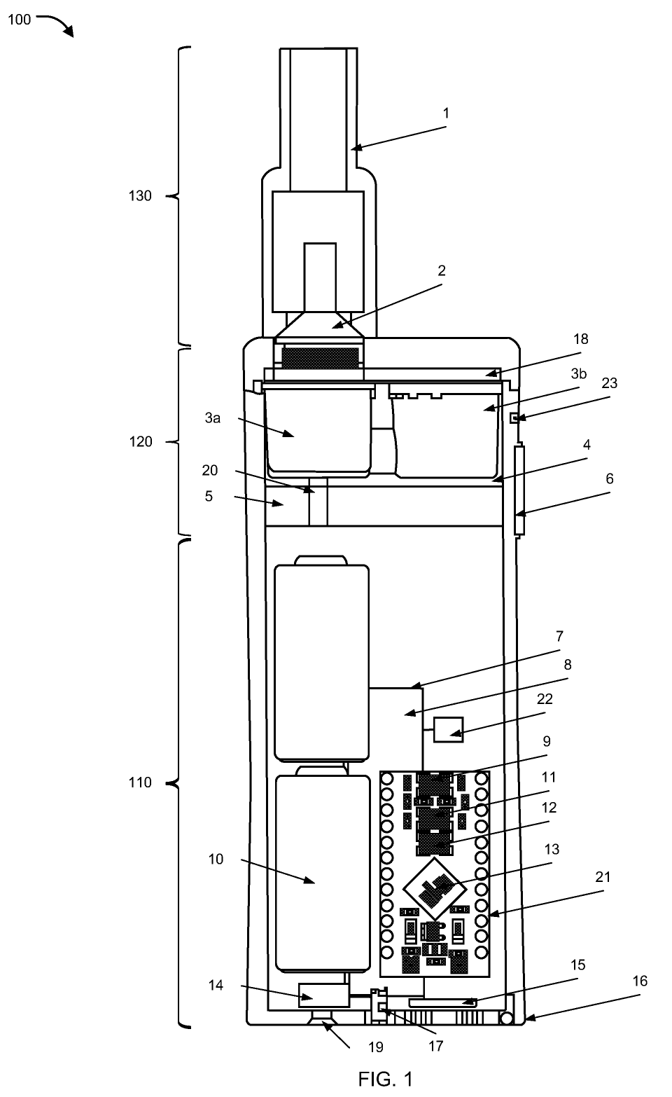

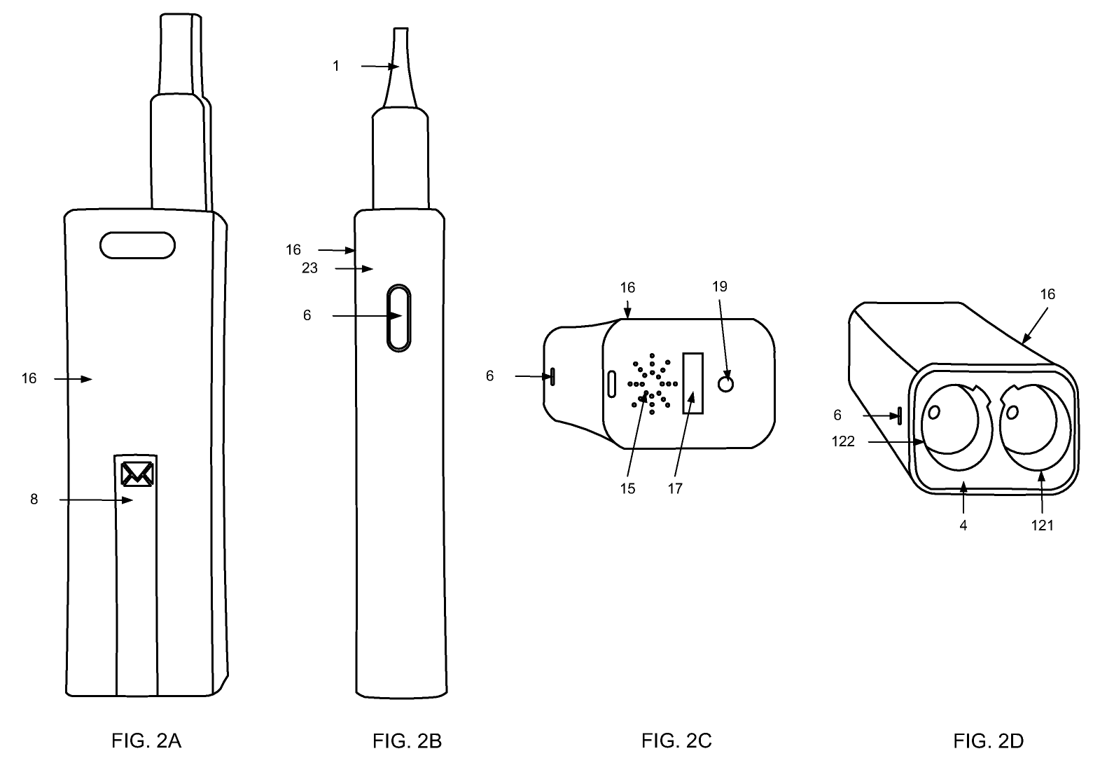

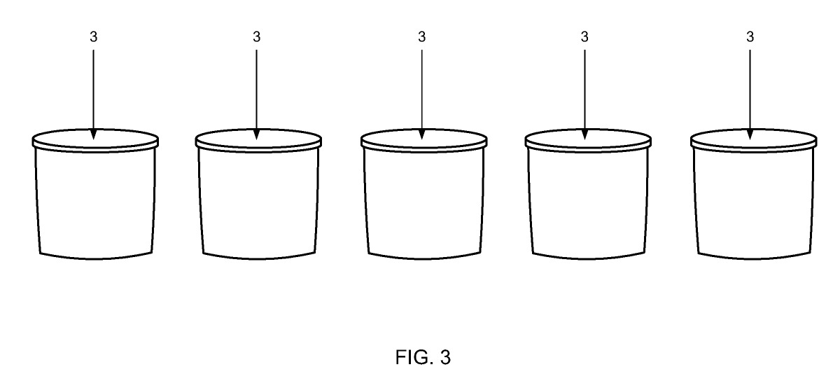

Referring to FIG. 1, there is depicted an inhalation device 100 adapted for delivering a pre-determined amount of a substance to a user by the inhalation of a vapor containing the substance. As shown in FIG. 1, the device 100 includes three main components: an electronics section 110, a vaporizing section 120, and an inhaler section 130. In the illustrated embodiment, the vaporizing section 120 and the electronics section 110 can be contained in a housing 16 (alternately “casing 16”). The housing 16 can provide protection to the electronics within the electronics section 110 and the vaporizing section 120 from the environment. In an embodiment, the inhaler section 130 can removably project from the end of the device 100, housing the vaporizing section 120. When assembled, the device 100 can define an elongated, rectangular shape as shown in FIGS. 1 and 2A-D. As described in greater detail below, the vaporizing section 120 can be configured to receive one or more pod(s) 3, shown in FIG. 3, containing a substance (not shown).

As used herein, “drawing”, “inhaling” or “puffing” from the device 100 involves a user placing the mouthpiece 1 of the inhaler portion 130 into mouth and sucking in, or inhaling air (or vapor containing one or more substances) from the device 100. Drawing, inhaling, or puffing is typically done by mouth (but could also be done by nose) and can include, but does not necessarily require, the substance to enter the lungs. In an embodiment, the device 100 includes a vapor sensor (not shown) (e.g. an air volume meter) adapted to detect and/or measure the flow (including the composition) of air and/or vapor traveling through the device 100 (e.g. during an inhalation). In an embodiment, the vapor sensor (not shown) detects and/or analyzes the vapor and the components therein. The vapor sensor (not shown) can be positioned within the electronics section 110, the vaporizing section 120, and/or the inhaler section 130 for the detection and/or measurement of the flow of air and/or vapor through the device 100. In an embodiment, as shown in FIG. 1, the mouthpiece 1 is adapted to facilitate user inhalation. In some embodiments, the inhaler section 130 further comprises a cooling coil 2 to reduce the temperature of vapors exiting the vaporizing section 120 before entering the mouth of the user. Person skilled in the art will appreciate that any conventional cooling coil 2 configured for personal inhalation devices may be used in the inhaler section 130. The mouthpiece 1 may be disposable or reusable.

As shown in FIG. 1, the vaporizing section 120 comprises a heating chamber 4 (or alternately a “vaporizing chamber 4”) that can be configured to receive one or more pods 3; persons skilled in the art, however, will understand that the chamber 4 may be configured to receive one or more pods. The heating chamber 4 can comprise a heater 5. The heater 5, which can be a ceramic heater, Kapton heater, laser, induction heater, and/or convection heater, can be configured to increase the temperature of the vaporizing chamber 4 to vaporize at least a portion of the material present in the pod—for example, the liquid portion containing the substance—to form a vapor containing the substance for inhalation by the user. In operation, the heater 5 increases the temperature of the heating chamber 4 to a predetermined value between 100 to about 230 degrees Celsius or preferably between 120 degrees Celsius to about 220 degrees Celsius. The heater 5 can be adapted to achieve the desired temperature either instantaneously or over a predetermined period of time (e.g. in several stages). A staged temperature change can ensure that certain substances (e.g. cannabinoids) are effectively vaporized as different substances may vaporize at different temperatures. In an embodiment, a cooling coil 2 reduces the temperature of the vapor to about 100 degrees Celsius. In alternate embodiments, the mouthpiece 1 does not comprise a cooling coil 2 and vapor is cooled by adjusting the distance of travel between the heating chamber 4 and the mouthpiece 1.

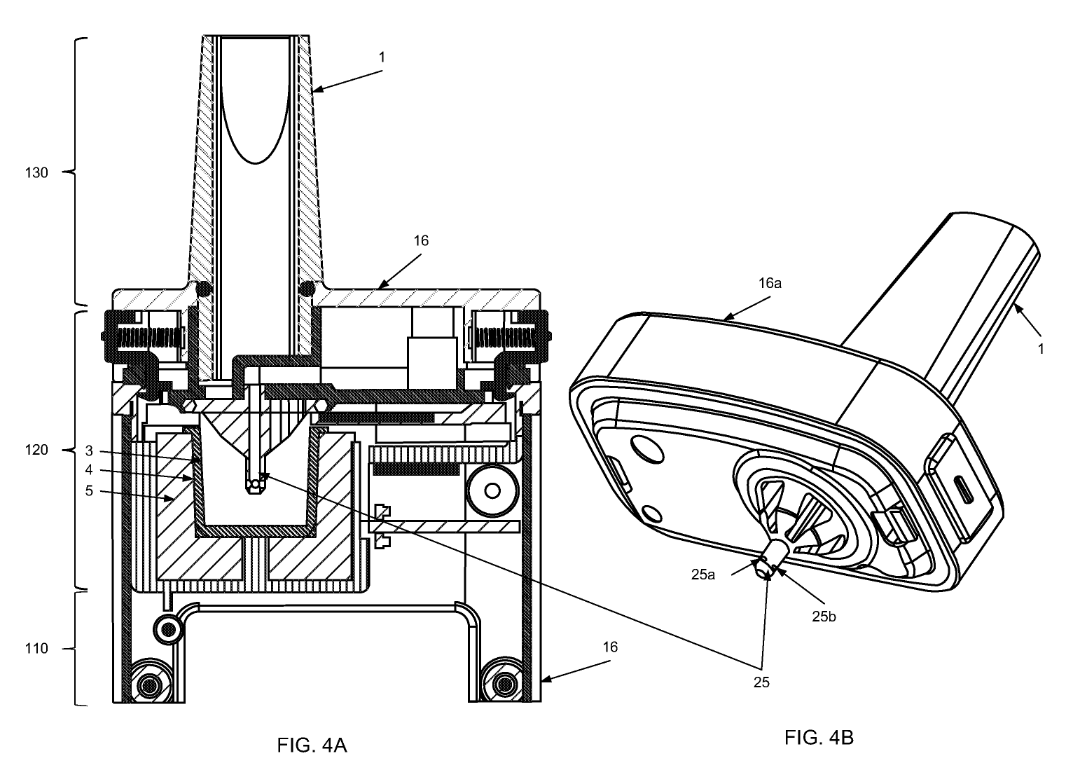

In an embodiment, the chamber 120 comprises a removable cover 16a (as best shown in FIG. 4) to facilitate insertion of new pod(s) 3 and disposal of used pod(s) 3. The cover 16a can be attached to the housing 16 using magnets. Persons skilled in the art, however, will understand that other means, including but not limited to a friction fit, resilient members or tabs, may be used to releasably secure the cover 16a to the housing 16. The removable cover 16a may optionally be integrated with the inhaler section 130. In an embodiment, the chamber 120 can be insulated by any means known to persons skilled in the art to maintain the temperature of the chamber 120, so that the device 100 may be handled by the user, and to insulate the electronics section 110 and/or other electronic components exterior to the electronics section 110 (e.g. reader 18). In an embodiments, the cover 16a comprises a cover sensor (not shown) to determine whether the cover 16a is properly closed. Using the cover sensor, the heater 5 can be configured to only activate when the cover 16a is securely fastened.

In an embodiment, as shown in FIG. 1 and FIG. 2, the chamber 4 comprises a first pod receptacle 121 and a second pod receptacle 122 for receiving a first pod 3a and a second pod 3b, respectively. When a pod 3a is inserted into the first pod receptacle 121, a surface of the pod 3a can be pierced by a perforator 25. The perforator 25, as best depicted in FIGS. 4A-B, can comprise multiple channels which allow air to enter and exit the pod 3a. In an embodiment, the heater 5 is proximal to the first pod receptacle 121. The second pod receptacle 122 is adapted to store another pod 3b for subsequent use (for example, another dose of the substance). In an alternative embodiment, the pod 3b in the second pod receptacle 122 may be adapted for concurrent use with the pod 3a in the first pod receptacle 121. In such a configuration, the device 100 is configured with two perforators and will heat both pods 3a and 3b according to their appropriate temperatures.

As shown in FIGS. 4A-B, the perforator 25 can include an intake channel 25a (alternately “an injection channel 25a) to facilitate the movement of air into the interior of the pod 3 and an exhaust channel 25b to facilitate the movement of air or vapor out from the interior of the pod 3. In some embodiments, the tube 20 may directly facilitate the movement of air into the pod 3. In an embodiment, the tube 20 facilitates the movement of air into a channel 25a of the perforator 25. Alternatively, in a dual chamber configuration, a bridge 26 (not shown) facilitates the movement of air from the exhaust channel of the first pod to an intake channel of the second pod (not shown). The perforator 25 can be configured to pierce, or enter, the pod 3 when the user applies a low level of force to the cover 16a. In addition, the perforator 25 can be adapted to create a seal after it pierces (or enters) the pod 3 to facilitate the efficient movement of air into and out of the pod 3. In an embodiment, the exhaust channel 25b comprises one or more filters (not shown) to reduce or eliminate the potential for substances in solid form (e.g. ground herbal medicine) to enter the exhaust channel 25b and be inhaled by the user.

Referring to FIG. 1, the housing 16 comprises an intake port 19 for drawing in air external to the device 100. An intake tube 20 can extend from the intake port 19 to the vaporizing chamber 4. Upon inhalation, air can be drawn into the intake port 19, passing through the intake tube 20 to the pod contained within the vaporizing chamber 4 via the intake channel 25a (as depicted in FIGS. 4A-B). Vapor containing the substance is inhaled by the user via the exhaust channel 25b (as depicted in FIGS. 4A-B) to an orifice formed in the mouthpiece 1 of the inhaler section 130.

The operation of the device 100 is controlled (e.g. heating rate and temperature) by micro-electronic components (hereinafter referred to as the “control electronics”) contained in the electronics section 110. The device 100 and control electronics are activated by toggling a power switch 6, as shown in FIGS. 1 and 2B. Referring to FIG. 1, the electronics section 110 includes an electronic circuit board 21 that contains control electronics for controlling the operation of, and collecting information from, the device 100. Referring to FIG. 1, the control electronics include a memory 12, connected to a logic circuit 13 (or alternately “processor 13”). The processor 13 is also connected to the heater 5, a graphical user interface 8 (or alternately “GUI 8”), a receiver/transmitter 9, a gyroscope/accelerometer 11, a vibrational motor 14, a speaker 15, a reader 18, a device database 22, a microphone 23, a vapor sensor (not shown) and/or temperature sensor (not shown). The control electronics can be electrically connected to a power source 10 (e.g. rechargeable lithium ion batteries) in any suitable manner, such as wiring (not shown).

The control electronics can be programmable (as described in greater detail below) such that a user is able to selectively determine the operation of the device 100. In an embodiment, the GUI 8 is used for programming the control electronics. In addition to the GUI 8, one or more input devices (not shown) (e.g. a smartphone, a tablet, etc.) may provide an additional user interface (e.g. via an application) for programming the control electronics, allowing the user to select an operating mode, and/or otherwise set or change operating parameters of the device 100. Programming instructions are either embedded in the processor 13 or stored in the memory 12 with the desired programming being accessed for use from the processor 13 by manipulating one or more of the input devices (not shown).

Programming instructions may also be loaded onto a general purpose computer, a special purpose computer, or other programmable data processing apparatus, such that the instructions that execute on the computer or other programmable data processing apparatus create means for implementing one or more functions specified herein. These computer program instructions may also be stored in a local (or remote) database or computer-readable memory that can direct a computer or other programmable data processing apparatus to function in a particular manner, such that the instructions stored in the local (or remote) database or computer-readable memory produce an article of manufacture including instruction means which implement the one or more functions specified herein. The computer program instructions may also be loaded onto a computer or other programmable data processing apparatus to cause a series of operational steps to be performed on the computer or other programmable apparatus to produce a computer-implemented process such that the instructions which execute on the computer or other programmable apparatus provide steps for implementing the one or more functions of the present invention.

In some embodiments, programming instructions can also be inputted into the device 100 via one or more input devices (not shown) or using the GUI 8. Persons skilled in the art will understand the processor 13 and the memory 12 (e.g. flash memory) can facilitate data processing and storage of programming instructions.

In some embodiments, the device 100 may be connected to an external device (not shown) via a programming port 17 that is configured to receive a cable, wire or the like for connecting the device 100 to the external device, such as a computer (not shown), smart phone (not shown), smart watch (not shown), and/or tablet (not shown). The programming port 17 is electrically connected to the processor 13 so that programming instructions can be provided to the processor 13 via the external device. The programming port 17 can be used to transfer data from the device 100 and recharge the power source 10.

Alternatively, any transfer of data between the device 100 and an external device—including transfer of data to the device (e.g. programming instructions) and from the device (e.g. user data)—may occur wirelessly using a wireless transmitter/receiver 9 disposed inside the electronics section 110 and electrically connected to the processor 13 (e.g. the Bluetooth™ proprietary open wireless technology standard which is managed by the Bluetooth Special Interest Group of Kirkland, Wash.). The user would then use an external device (e.g. a smartphone, smart watch, tablet, laptop, etc.) with compatible wireless capability to wirelessly transmit programming instructions to the device 100 or receive data from the device 100.

In an embodiment, data collected by the device—e.g. user data—is transmitted or transferred to a remote database and/or analysis unit. The remote database may or may not be located centrally relative to the device 100. A “remote database” may refer to one or more congruent and/or distributed databases, such as, for example, also including one or more sets of congruently inter-related databases. According to one or more embodiments of the present disclosure, the database(s) are provided remotely of the device 100.

This remote database can constantly receive collected user data from one or more devices 100 and store the collected user data in the database. The collected user data can be subjected to analysis that can involve the application of different algorithms to detect trends and/or patterns within the collected data. Analysis of the collected data may be performed by an external device and/or the analysis unit. Persons skilled in the art, however, will appreciate that the analysis of user data can be performed local to the device 100. The collected data can be analyzed for additional information associated with the user, user therapy, the pod(s), the device, the vaporized substance, trends amongst the users, trends in relation to the population, and/or trends in relation to one or more references.

A person skilled in the relevant art would generally understand that a reference to “Internet of Things (IoT)” refers to networked or interconnected objects, typically, but not limited to, everyday objects, more technically purposed objects (i.e. medical devices) and devices. It is described as a self-configuring wireless network of sensors whose purpose would be to interconnect all such connected devices. The concept is attributed to the former Auto-ID Center, founded in 1999, based at the time at the Massachusetts Institute of Technology (“MIT”).

In some embodiments, the pod 3 contains instructions for programming the control electronics of the device 100. For example, the interchangeable pod(s) 3 may be provided with a pod memory (not shown), a pod processor (not shown) and/or machine readable code (e.g. barcodes, Near Field Communication, etc.; not shown) containing programming instructions. Different pods 3 may provide different programming instructions. The programming of the device could, for example, be changed by installing different pods 3. That is, the desired programming may be achieved by installing the pod 3 having the appropriate programming instructions.

In one embodiment, the processor 13 controls the time period during which the heater 5 is active in order to control the rate of delivery and/or amount of the substance delivered by a single inhalation to the user. The control electronics activate the heater 5 when the power switch 6 is in the “on” position or based on pre-determined time intervals. In an alternative embodiment, the heater 5 is adapted to achieve the desired temperature instantaneously (preferably, but need not necessarily, within one second) of the user depressing an activator or button. When the user releases the button, the heater 5 turns off. While activated, the heater 5 vaporizes the desired substance creating a vapor containing the substance. The heater 5 ceases to vaporize the substance once deactivated (e.g. outside of a pre-determined time interval, when the power switch 6 is in the “off’ position, or when the activator is no longer depressed). Therefore, the amount of substance vaporized during a single inhalation may be limited by a pre-determined period of time that the heater 5 is activated. The duration of the time that the heater 5 is activated can dictate the maximum possible dosage of substance delivered by an inhalation regardless of how long air is drawn through the device 100 by the user during the inhalation. In some alternate embodiments, the device 100 is adapted to use the vapor sensor to detect the user inhaling or drawing on the device 100 and does not require that the user depress the activator or button to turn on the device 100.

Alternatively, it may also be possible to control the dose of the substance delivered by a single inhalation based on temperature rather than on time. In this case, a temperature sensor (not shown) configured to measure temperature within the vaporizing chamber 4 is electrically connected to the processor 13. When the heater 5 is activated, the substance is heated and vaporized, creating a vapor containing the substance. If the temperature in the chamber 4 exceeds a predetermined temperature value, the processor 13 deactivates the heater 5 limiting the maximum dosage of the substance available in an inhalation.

There are also multiple approaches for the processor 13 to control the maximum number of doses of substance discharged during a set period of time. For example, the processor 13 can set a minimum time interval between activations of the heater 5. That is, after the processor 13 activates the heater 5 in response to the user's inhalation on the device 100, the processor 13 will not activate the heater 5 again until a predetermined period of time (i.e. a minimum time interval) has elapsed. If the user inhales again on the device 100 before the minimum time interval has elapsed, the processor 13 will not activate the heater 5. The minimum time interval can be set by the user via one or more of the input devices (not shown), the GUI 8, or may be programmed into a pod 3.

The user can re-program the control electronics using the GUI 8 or one or more of the external input devices to change the maximum dosage discharged with a single inhalation and/or the maximum number of doses of the substance that can be discharged during a set period of time. Programming to control the dose of substance delivered can be based on factors such as the type of substance (i.e. the type of herb) or the amount of the substance contained in the pods 3 being used. In some embodiments, the programming to control dose is a matter of the user's individual preferences. In other instances, the user's ability to change or program the device 100 can be restricted based on regulatory, administrator, and/or healthcare provider limits.

The GUI 8 can utilize any suitable display technology, including but not limited to LED, LCD, E-Ink. The GUI 8 can be configured for direct user interaction (e.g. touchscreen). The GUI 8 can be used for displaying a variety of information to the user—collectively user data. Examples of information (or user data) that can be collected by the device 100 and displayed by the GUI 8 include, but are not limited to, the type of pod, the dosage, battery status, time of day, instructions for using the device, type of substance being vaporized, number of remaining doses, current count of dosages, time-out period and other metering feedback. In addition, the user may input certain user information into the device 100 using the GUI 8 or external device including, but not limited to, the number of doses, symptoms and symptom strength, side effects, and patient notes (e.g. treatment effectiveness) which may be stored in the device database 22 as user data. In some embodiments, the device 100 may be configured to prompt or query the user to provide some or all of the above user information at pre-determined intervals (e.g. after each inhalation).

User data collected by the device 100 may be used for various applications, including but not limited to allowing healthcare providers to enhance user treatment by analysing the user data and implementing appropriate strategies to enhance the efficacy of the substance or overall treatment. Thus, the device 100 allows a healthcare provider to enhance user outcomes. User data may be used in a clinical setting to determine efficacy and other treatment variables on a more generalized basis (e.g. as anonymous population data). User data can also be used to enhance individual user outcomes. For example, physicians can provide users with a sample of an herbal and/or therapeutic substance and obtain feedback (i.e. user data) from the device 100 to allow the physician to evaluate the effectiveness of the substance. Additional exemplary applications of the method and device 100 of the present disclosure may include: (a) pain relief and other symptomatic relief for medical cannabis patients; (b) naturopath and other alternative health care practitioner prescribed herbal remedies for symptomatic relief; (c) sleep support, anti-emetic, mood control by vaporizing herbs and related essential oils; and/or (d) a research tool for major universities and pharmaceutical companies to be used for testing alternative delivery mechanisms for medicine.

In some embodiments, the inhaled substance is derived from cannabis or marijuana plant. The device 100 can vaporize the bioactive components (e.g. cannabinoids) of the marijuana plant (e.g. present in the form of cut up plant matter, ground/powderized plant material comprising bud, leaf and stem portions, etc.) while avoiding any burning of plant matter.

The control electronics can be programmed to retain device operation information, or user data, using the device database 22 in real-time or subsequent download over a synchronization channel. The user data collected may also include the number of inhalations per day, the number of inhalations per minute, the number and types of pods used by the user, the average dosage in a given time period, etc. The collected user data can be stored in the memory 12 or device database 22 and transferable to another device and/or application in any suitable manner. User data may be transferred wirelessly, for example, to a remote database, a third party server, and/or a cloud-based application. Alternatively, the port 17 could be used to connect the device 100 to an external device, such as a computer, with a USB (micro-USB) cable or the like to transfer the user data to the external device. The user data transfer can allow users or other third parties to analyze the usage of the device 100 (e.g. a physician could monitor a patient's use of medical marijuana). Persons of skill in the art will also understand that data transferred to an external device may subsequently be transferred to a remote database or third party server for additional analyses. Also, in some embodiments, user data can be transferred from the device 100 as it is collected or at a time following the collection of the date (e.g. when the device 100 is in communication with the external device and/or remote database).

As described above, each pod 3 is sealed and contains one or more substances, preferably a dry herb that is in the form of a powder, has been cut up, or remains intact. For example, all or part of a cannabis, or marijuana, plant (that has been powderized, cut into a plurality of pieces, or left intact) may be loaded into a pod 3 to provide a source for various substances, including but not limited to, terpenoids and cannabinoids (e.g. tetrahydrocannabinol, cannabidiol and cannabinol). Other substances that may be used with the present disclosure include, but are not limited to, caffeine, medications, vitamins, herbs, mushrooms and nicotine. As shown in FIG. 3, the pods 3 can maintain the substance in a sterile environment and, when used with the device 100 of the present disclosure, provide metered or controlled doses of the substance.

In operation, a user inhales vapor containing one or more substances from the device 100 by puffing on the mouthpiece 1 of the inhalation section 130. This causes external air to be drawn into the device 100 via the port 19. Specifically, external air is drawn in through the port 19, passes through the tube 20, through the intake channel 25a of the perforator 25 and into the interior of the pod 3 that is loaded in the vaporizing chamber 4. The device 1 can be activated when the power is turned on, the app (for example, loaded on an external device such as a smartphone, smart watch or tablet) is turned on or when a pod is placed near the device 1. The substance can be heated and vaporized when the user, for example, depresses the activator. The inhaled air mixes with the vapor in the pod 3 (vapor containing the substance) and carries the vapor though the exhaust channel 25b of the perforator 25 and into the inhalation section 130, wherein the vapor is cooled by the cooling coil 2 (as shown in FIG. 1), before transiting through the mouthpiece 1 and into the mouth of the user. Each instance of the user drawing or puffing on the device 100 is referred to herein as an inhalation. Following each inhalation, or series of inhalations, the user can input (e.g. manually via the GUI, via gestures, voice, etc.) how he/she is feeling.

In another embodiment, the vaporizing chamber 4 comprises a pod 3 wherein dry herb that is in the form of a powder, has been cut up, or remains intact, may be loaded into the chamber 4. In another embodiment, a pod 3 can comprise multiple chambers containing one or more substances of varying strength/potency, flavor or other characteristics as may be desired by the user. The pods 3 can be associated with a machine readable code (e.g. a barcode, RFID or NFC on a surface of the pod 3) that may be read by the reader 18. The code may be configured so that, upon scanning by the reader 18, the processor 13 recognizes various characteristics of the pod 3, including but not limited to the substance contained (and the temperature required to vaporize the substance), the dosage of the substance, the manufacturer, the expiration date. For example, if the pod 3 is prepared by an unrecognized manufacturer, or if the pod 3 is expired, the device 100 may not operate and/or an alert may be generated for the user.

As described above, the device 100 comprises a GUI 8, a gyroscope/accelerometer 11, a vibrational motor 14, a speaker 15, a reader 18 and/or a microphone 23 for collecting and/or presenting user data to the user. In some embodiments, the GUI 8, vibrational motor 14 and speaker 15 may also be used to generate alerts for the user. In accordance with the present disclosure, the user can use gestures (e.g. direction of device 100 to indicate his/her feeling or status) in order to provide feedback to the device 100 (e.g. user data).

The system of the present disclosure is configured for use with a communication network. The communication network may include satellite networks (e.g. GPS), terrestrial wireless networks, and the Internet. The communication of data between a device subsystem, an external device subsystem and/or an accessory device subsystem may also be achieved via one or more wired means of transmission (e.g. docking the device 100 in a base station of the external device subsystem), or other physical means (e.g. a Universal Serial Bus cable and/or flash drive) of transmission. Persons having ordinary skill in the art will appreciate that the system includes hardware and software.

In an embodiment, the device subsystem may comprise a hardware and/or software application that allows for the receipt or transmission of data that has the capability to use the 802.11 protocol, Bluetooth communication and/or another linkage. For example, cellular communication and/or the communication network may be used. Additional hardware and/or software applications may: (i) be enacted upon associating the device with a user; (ii) connect wirelessly to one or more processor(s) of an external device (e.g. via Bluetooth, Wi-Fi and/or another linkage) for the exchange of data; and/or (iii) store data (e.g. user data) in one or more databases for subsequent transmission and/or analysis.

In an embodiment, the device 100 delivers desired substance(s) via vaporization by accommodating measured, pre-packaged dose(s) of the desired substance in pod(s) and placing the pod(s) in a specially-designed heating chamber for inhalation by a user.

In an embodiment, the device is also configured to collect user data including, among other information, user status, time, date and number of doses administered. By collecting user data, the device enables analysis and control of therapy by authorized parties (e.g. healthcare professionals, law enforcement, governments, etc.). In one embodiment, the heating chamber receives pod(s) containing a pre-packaged dose of the substance(s) for vaporization, and the device is configured to collect user data and to interface with external devices and/or remote databases.

In an embodiment of the present disclosure, the device, system and/or method provides advantages for patients, healthcare professionals, regulators, and/or administrators. Device users can be provided with an easy-to-use, safe and healthier alternative to smoking, in addition to a precise, consistent, and/or reliable means for administering substances (e.g. medical cannabis) to optimize therapeutic benefits.

In an embodiment of the present disclosure, the device, system and/or method can produce a mild, non-irritating and non-noxious vapor. The use of pre-packaged pods containing a predetermined dose of the substance for vaporization allows for improved delivery of active ingredients (i.e. improved bioavailability of the substance and/or therapeutic efficacy).

Breath Analysis

In an alternate embodiment, the device 1 may be used to analyze the breath of a user to determine the amount of cannabinoids (or any one or more bioactive components of the cannabis plant) the user has absorbed (e.g. bioavailability). In operation, the user would blow into the device 1 and the vapor sensor (not shown) would detect any cannabinoids in the incoming air flow.

Digital Health Infrastructure

In an embodiment, the present disclosure further comprises a digital health infrastructure (“DHI”). The DHI can be a cloud-based application adapted for communication with the device. The DHI is configured to receive, store, and/or analyze user data, including symptoms, pain relief, appetite, physical energy, exercise, diet, and use of (including therapeutic and adverse effects of) other medications. The user data is securely encrypted with controlled access.

In some embodiments, the DHI provides healthcare professionals with a comprehensive report generated based on the user data collected by the device and/or data from external devices (including third party monitoring devices such as smart blood pressure machine, glucose monitoring, etc.).

The DHI can be compatible with various operating systems including, but not limited to iOS, Android, Windows, and Internet web browsers (e.g. Internet Explorer, Firefox, etc.).

The DHI can conduct a variety of analyses (and creates a number of reports) based on the user data and/or data from external devices including, but not limited to: Dosing—measures and/or monitors dosing for users; Dose effective tracking—collects user feedback after each dose; Symptom tracking—monitors all patient symptoms; Medication tracking—monitors all medications that patients are using or have used; Reporting—creates graphs and trend chart(s) and easy to comprehend infographics on patient(s) or users and may be compared with patient(s) or users with similar health issues; Lifestyle program—provides a unique lifestyle program and measures patient involvement and results; Retail—linked into the retail environment to ensure that patients can acquire refills and new products; Supply chain management—links into cultivation network to ensure that growers and distributors have the information they need to supply patients with the best products; and/or Support—allows patients or users to connect to the people most important to them and encourages interaction and support.

In an embodiment, the DHI collects data, including user data, on: all of the medications taken; the effectiveness of each dose; how a patient or user feels; exercise; diet; mindfulness; support; and information from other, or external, devices (e.g. weight, blood pressure, blood sugar, cholesterol, etc.).

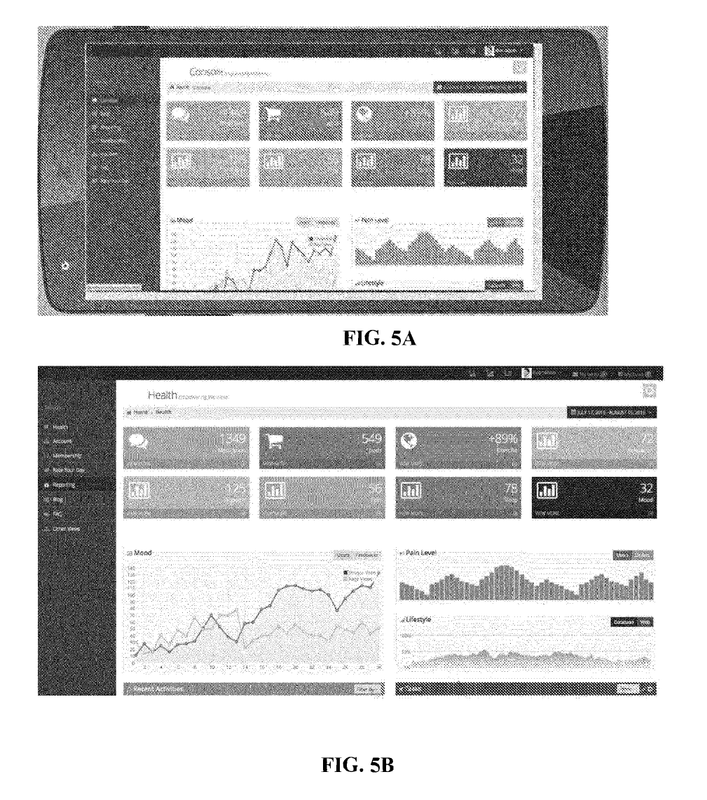

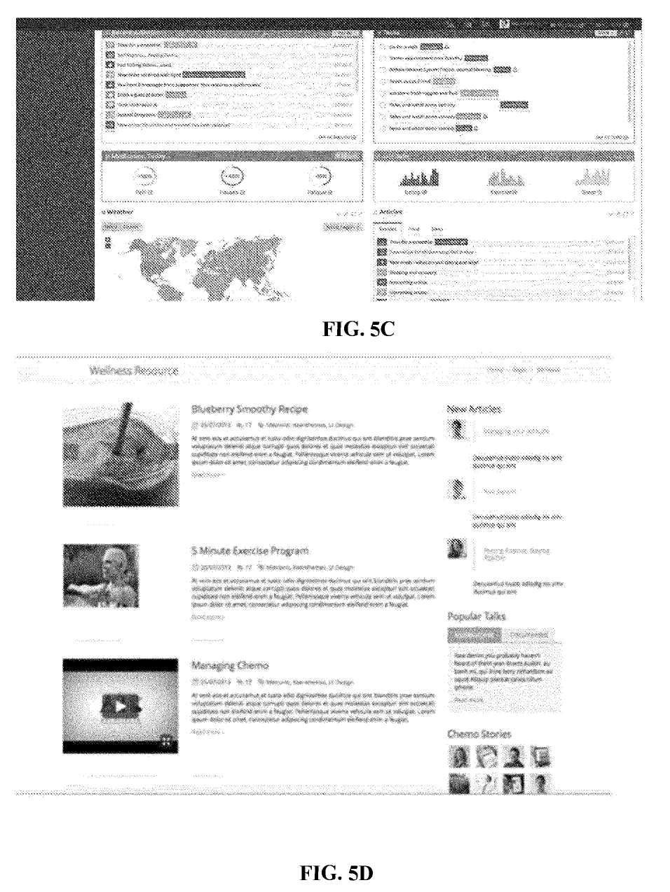

FIG. 5A shows the DHI console or homepage, in accordance with an embodiment of the present disclosure, which includes, among other features, information about medication, retail, exercise, relaxation, support, pain, sleep, and mood.

FIG. 5B depicts, in accordance with an embodiment of the present disclosure, a sample DHI report, which includes, information on mood trends, pain level trends, and lifestyle trends.

FIG. 5C depicts, in accordance with an embodiment of the present disclosure, user information on recent activities, tasks, medication (e.g. pain, nausea, fatigue), lifestyle (e.g. eating, exercise, sleep), weather and articles.

FIG. 5D shows, in accordance with an embodiment of the present disclosure, sample wellness resources for users (e.g. smoothie recipes, exercise programs, managing medications, etc.).

While the foregoing describes various embodiments of the disclosure, other and further embodiments of the disclosure may be devised without departing from the basic scope thereof. The scope of the invention is determined by the claims that follow. The invention is not limited to the described embodiments, versions or examples, which are included to enable a person having ordinary skill in the art to make and use the invention when combined with information and knowledge available to the person having ordinary skill in the art.

### ADVANTAGES

The present disclosure provides an inhalation device that can assist self-medicating users in determining the effect of medication outside of physician's direct observation.

The present disclosure provides an inhalation device that can collect and disseminate valuable patient information to third parties for further research and development in inhalable medications.

The present disclosure provides an inhalation device that can deliver sterile substances.

The present disclosure provides an inhalation device that can render information about the amount of vaporized substance inhaled by a user.

The present disclosure provides an inhalation device that can monitor a user during different stages of inhalation.

The present disclosure provides an efficient and cost-effective inhalation device.

I/We claim:

1. A device for use in association with a container containing a substance for delivery to a user, the device comprising:
a vaporizing section adapted to receive the container and vaporize the substance contained therewithin;
an inhalation section adapted to deliver the vaporized substance to the user; and
an electronics section configured to perform (a) controlling operation of the device, and (ii) generating user data associated with the delivery of the vaporized substance to the user.

2. The device of claim 1, wherein the vaporizing section comprises a heater for vaporizing the substance.

3. The device of claim 1, wherein the vaporizing section is adapted to receive at least two containers.

4. The device of claim 1, wherein the device further comprises a vapor sensor for detecting and/or measuring composition and flow of air and/or vapor moving through the device.

5. The device of claim 1, wherein the inhalation section further comprises a cooling coil for decreasing vapor temperature.

6. The device of claim 1, wherein the heater is configured to achieve a predetermined temperature either instantaneously or over a predetermined period of time.

7. The device of claim 1, wherein any or a combination of the vaporizing section and the inhalation section further comprise(s) a perforator to facilitate air circulation within the container.

8. The device of claim 1, wherein said device further comprises an intake port for drawing-in air external to the device.

9. The device of claim 1, wherein said container is a pod.

10. A system for delivery of a substance contained in a container to a user, the system comprising:
at least one device for vaporizing the substance contained in the container, wherein said at least one device comprises at least one processor operative to effect any or a combination of (a) controlling vaporization rate of the substance and (ii) collecting and transmitting user data associated with delivery of the vaporized substance; and
a database configured to electronically store the user data associated with delivery of the vaporized substance.

11. The system of claim 10, wherein the at least one device further comprises a geographic tracking means operative to obtain location data of the user.

12. The system of claim 10, wherein the at least one device further comprises any or a combination of an accelerometer and a gyroscope to measure and/or determine position of the device and to automatically associate accelerometer data and/or gyroscope data with the device and/or the user.

13. The system of claim 10, wherein the at least one device comprises a graphical user interface for presenting the user data.

14. The system of claim 10, wherein the processor is operative to electronically generate an alert.

15. The system of claim 10, wherein the system further comprises an external device for receiving user data from the device and/or sending external device data to the at least one device.

16. The system of claim 10, wherein the processor is operative to electronically generate a report based on the user data.

17. The system of claim 10, wherein the processor is operative to electronically control any or a combination of activation of the heater and temperature of the heater.

18. A system of claim 10, wherein the device further comprises a reader operative to read a machine readable code associated with the container to collect data associated therewith.

19. A system of claim 10, wherein said container is a pod.

20. A method for delivering a substance contained in a container to a user, said method comprising the steps of:
(a) loading said container into a device; and
(b) operating said device to effect vaporization of said substance contained in the container to enable delivery thereof to the user,
wherein, said device is operatively coupled to at least one processor, and wherein said at least one processor is configured to (i) control vaporization of the substance and (ii) collect and/or transmit data associated with the delivery of the vaporized substance to the user.
